(19) United States
(12) Patent Application Publication
(10) Pub. No.: US 2015/0313954 A1
(43) Pub. Date: Nov. 5, 2015

(54) METHOD OF ADMINISTRATING A SERIES OF COMPOSITIONS FOR DYNAMIC IMMUNE MODULATION

(71) Applicants: Pichaet WIRIYACHITRA, Bangkok (TH); Ampai PANTHONG, Bangkok (TH); Watchara KASINRERK, Chiang Mai (TH); Sumitra THONG-PRASERT, Chiang Mai (TH); Sirithip WIRIYACHITRA, Bangkok (TH)

(72) Inventors: Pichaet WIRIYACHITRA, Bangkok (TH); Ampai PANTHONG, Bangkok (TH); Watchara KASINRERK, Chiang Mai (TH); Sumitra THONG-PRASERT, Chiang Mai (TH); Sirithip WIRIYACHITRA, Bangkok (TH)

(21) Appl. No.: 14/418,714

(22) PCT Filed: Aug. 1, 2012
(86) PCT No.: PCT/TH2012/000032

§ 371 Date: Jul. 10, 2015

(51) Int. Cl. A61K 36/61; A61K 36/48; A61K 36/185; A61K 36/23; A61K 36/38

(52) U.S. Cl. CPC A61K 36/61; A61K 36/23; A61K 36/38; A61K 36/185; A61K 36/48

(57) ABSTRACT

The present disclosure provides a series of compositions, or a single composition formulation (e.g., which includes distinct time release portions), supportive of or resulting in antipode immune system effects. A method of administrating the series of compositions or the single composition formulation to support, provide, enhance, or improve health benefit by way of modulating aspects immune system function is also provided.

### TECHNICAL FIELD

The present disclosure relates generally to a series of compositions or a single composition formulation (e.g., which includes distinct time release portions) supportive of or resulting in antipode immune system effects, and a method of administrating the series of compositions or the single composition formulation to support, provide, enhance, or improve health benefit by way of modulating aspects immune system function, for instance, to support a favorable immune system response to a health condition such as cancer or autoimmune disease.

### BACKGROUND

White blood cells (WBCs), or leukocytes, are a part of immune system, particularly the adaptive immune system. White blood cells are essential for fighting pathogens, for example, bacteria, viruses, and other foreign matters that invade the body of living organisms causing infection and diseases. Elevated WBCs are a sign of an infection.

In a normal adult body there are approximately 4,000 to 10,000 (average 7,000) WBCs per microliter of blood or approximately 20,000-55,000 million white blood cells in a healthy male. White blood cells can be categorized into three groups according to their functions, namely: 1) phagocyte, NK (natural killer) cells and cytotoxic T-cells; 2) B-cell and plasma cells; and, 3) Tₕ or T-helper cells (which stimulate immune reactive processes). The amount of each type of white blood cell within the body must be regulated or modulated to maintain bodily homeostasis. Imbalance of these cells manifests in, for example, autoimmune disease (where body exhibits elevated WBC levels and attacks itself) and leucopenia (where the body exhibits reduced levels of WBC making it vulnerable to infection).

Cancer is an umbrella term that refers to approximately 200 diseases that possess two common characteristics namely: 1) an uncontrolled growth of cells; and, 2) an ability to invade and damage normal tissues either locally or at distant sites in the body. The immune system is of vital importance for recognizing and eliminating malignant tumors and lessening the effects of cytotoxic (cell-killing) therapy such as chemotherapy. Many chemotherapeutic treatments act by suppressing the immune system followed by intensive antineoplastic treatment. This creates a two-fold detriment to the body: 1) the body's natural defenses (immune system) are suppressed leaving the body open to attack from other pathogenic organisms and preventing the body from fighting off ancillary mutations or cell proliferation that can occur; and, 2) common antineoplastic agents are broad acting, and destroy both healthy and cancerous tissue. Thus, cancer treatment and cure is the subject of ongoing research.

One of the research aspects is the role of the immune system in cancer pathogenicity and pathology. Several white blood cell populations have been shown to affect cancer cells, including T_{reg} (which down-regulate the immune system and help in identification of “self” cells as opposed to foreign matter) and Tₕ-17. However, in view of standard methods of cancer treatment, including immune suppression followed by antineoplastic treatment, methods of adequately modulating the activity of the immune system while simultaneously suppressing and/or reducing cancer or tumor progression have presented a challenge.

Additionally, simultaneously achieving a proper modulation or regulation of numbers of each type of immune cell within the body without inducing an autoimmune type reaction is also problematic. For instance, when a given particular drug, pharmaceutical composition, or substance is introduced to a body of living organism, such a drug, composition, or substance can effectuate an increase in number of one type of white blood to boost the immune system (e.g, Tₕ-1), and concurrently lead to excess or imbalance in number of another type of immune cell (e.g. Tₕ-2).

Hence, a need exists for a novel composition capable of supporting or facilitating the modulation or regulation of immune cells for improving public health, particularly for supporting an immune system response to diseases such as cancer, while simultaneously supporting of facilitating cancer cell reduction.

### SUMMARY

In accordance with a first aspect of the present disclosure, there is disclosed a method of supporting or providing fluctuating modulation to immune function including administering a set of direct immune modulating compositions; and administering an indirect immune modulating composition, wherein the administration of the set of direct immune modulating compositions and the administration of the indirect immune modulating composition is separated by about 2 to about 6 hours.

In accordance with a second aspect of the present disclosure, there is disclosed an immune modulating time-release compound including an immediate release portion comprising an indirect immune modulating composition; and at least one delayed release portion comprising a set of direct immune modulating compositions.

In accordance with a third aspect of the present disclosure, there is disclosed a kit including an indirect immune modulating composition in a first administration form; and a direct immune modulating composition in a second administration form, wherein the indirect immune modulating composition and the direct immune composition result in antipode effects upon an immune system of an organism to which the indirect immune modulating composition and the direct immune modulating composition are administered.

In accordance with a fourth aspect of the present disclosure, there is disclosed a method for reducing biomarker levels associated with cancer including administering an indirect immune modulating composition in the form of a liquid comprising xanthones at a first time point; and, administering a direct immune modulating composition in the form of capsules including Gotu Kola, and at least one of sesame, soybean, guava and mangosteen at a second time point; wherein the first time point and the second time point are separated by about 2 to about 6 hours.

The foregoing summary is illustrative only and is not intended to be in any way limiting. In addition to the illustrative aspects, embodiments, and features will become apparent by reference to the drawings and the following detailed description.

### BRIEF DESCRIPTION OF THE FIGURES

A description of embodiments of the present disclosure is provided below with reference to the figures, in which:

FIG. 1 is a representative graph illustrating an effect of a composition comprising an extract from mangosteen, Gotu Kola, sesame, soybean, and guava according to the present disclosure on the level of Tₕ-1, Tₕ-2, Tₕ-17, and T_{reg} cells; and

FIG. 2 is a representative graph illustrating an effect of a composition comprising an extract from mangosteen according to the present disclosure on the level of Tₕ-1, Tₕ-2, Tₕ-17, and T_{reg} cells.

### DETAILED DESCRIPTION

Unless explicitly stated otherwise, in the description herein, the recitation of particular numerical values or value ranges is taken to be a recitation of particular approximate numerical values or approximate value ranges. For instance, a given numerical value or value range provided below should be interpreted or defined as an approximate numerical value or value range (e.g. a quantity of 5 mg should be interpreted as approximately or about 5 mg).

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a composition, a composition constituent or ingredient, a physical parameter, a process step, or a value depending upon the type of set under consideration.

In the context of the present disclosure, the term phytochemical shall refer to any compound or chemical that occurs naturally in plants (i.e. living organisms belonging to the kingdom Plantae), or to any artificial or synthetic compound, composition, or chemical having an identical, almost identical, or significantly similar chemical, physical, and/or structural properties to that which occurs naturally in plants, and the term phytochemical composition shall refer to a composition including at least one phytochemical. Unless defined otherwise, all technical and scientific terms used herein have the same meanings as commonly understood by a person of ordinary skill in the relevant art of the present disclosure.

In the context of the present disclosure, the term living organism refers to human beings and animals (i.e., organisms belonging to the kingdom Animalia).

The term “immune modulating” as used herein can refer to increasing or supporting the increase of one or more cytokine levels, or decreasing or supporting the decrease of one or more cytokine levels, or providing components that support, lead to, or induce or facilitate tumor or cancer cell reduction by way of supporting or aiding the immune system.

Embodiments in accordance with the present disclosure are directed towards a set or series of compositions intended for supporting immune system modulation or function, and particularly for supporting an immune response to conditions such as cancer, by way of supporting, enhancing the likelihood of, and/or providing particular types of immunomodulatory effects. Embodiments in accordance with the present disclosure are further directed towards a procedure, technique, or method of administrating a set or series of compositions to provide, enhance, or improve health benefits relating to immune modulation or support. In embodiments, the immune modulation and immune system support can facilitate cancer cell reduction and/or reduce the severity of symptoms caused by allergic reactions (e.g. rhinitis, rash, congestion, abdominal distress, and the like) or autoimmune diseases (e.g. Lupus, rheumatoid arthritis, psoriasis, type-1 diabetes, Crohn's disease, and the like).

A set or series of compositions in accordance with an embodiment of the present disclosure can be administered to a living organism in predetermined amounts at predetermined times to form a protocol or regime that is expected to support, enhance, or improve the health of the organism. Further health support or benefit related to immune modulation, for example, cancer cell reduction or cytokine modulation, can accrue to the organism (e.g., over time) due to the administration of a set or series of compositions in accordance with the present disclosure.

Embodiments in accordance with the present disclosure support, lead to, induce, provide for, or result in immune modulation in such a manner so as to decrease the expression of particular 11 populations, followed by increasing the expression of certain Tₕ populations. More particularly, within a set or series of compositions, the administration of a first composition can support, lead to, induce, or provide an indirect immune response corresponding to decreasing or reducing the expression of particular Tₕ populations; and the administration of a second composition can support, lead to, induce, or provide a direct immune response corresponding to enhancing the expression of certain Tₕ populations. Thus, the administration of the first and second compositions in accordance with an appropriate immunomodulatory regime or protocol in accordance with an embodiment of the present disclosure can support, lead to, induce, or provide antipode immune system reactions or responses relative to each other.

The term “antipode” as used herein means that the effect of a composition (an “indirect immunomodulatory composition” or “indirect composition”) that supports or results in a decrease or reduction in an overall level of cytokines is substantially, generally, or proportionately opposite the effect of a composition (a “direct immunomodulatory composition” or “direct composition”) that supports or results in an increase or enhancement in an overall level of cytokines. Thus, an indirect composition supports or provides one or more effects that are antipode or substantially, generally, or proportionately opposite to the effect(s) supported or provided by a direct composition.

Immune modulating compositions in accordance with embodiments of the present disclosure can include or incorporate one or more components (e.g., phytochemical compositions and nutraceutical compositions). More particularly, the decreased expression of a Tₕ population supported or provided by an indirect composition is associated with administration or delivery of a substantial, moderately high, or high dose of one or more naturally occurring xanthones. The direct immune modulating composition, associated with increased or enhanced expression of a Tₕ population, can include at least an extract from Garcinia Mangostana L. (hereinafter referred to as “mangosteen”) and an extract from Centella Asiatica L. (hereinafter referred to as “Gotu Kola”). In accordance with embodiments of the present disclosure, the increased expression of a Tₕ population supported or provided by a direct composition is associated with administration or delivery of a synergistic combination of one or more naturally occurring xanthones and triterpenoid saponins.

In an embodiment, two or more immune modulating compositions (e.g., indirect and direct immune modulating compositions) are administered or delivered to an organism (e.g., a mammal), which can support, lead to, induce, or result in antipode reactions in the organism's immune system. In certain embodiments, a single time-release compound or delivery vehicle having both an immediate release portion (e.g., an indirect composition portion) and a delayed release portion (e.g., a direct composition portion) is administered to support or provide such immune modulating activity, wherein the immediate release portion of the composition and the delayed release portion of the composition support, lead to, induce, or result in antipode reactions in the immune system. In particular embodiments, a single time-release compound or delivery vehicle can include an indirect composition portion, which is intended for release or delivery substantially immediately or at an intended first time; and multiple direct composition portions, each of which is intended for release or delivery on a sequential or successive basis (e.g., at predetermined relative times) following the release or delivery of the direct composition portion (e.g., subsequent to the first time, and separated from the first time by a predetermined or expected average delay time or interval).

The indirect and direct immune modulating compositions support or stimulate antipode reactions in the immune system. These antipode reactions are referred to together herein as “fluctuating modulation” of the immune system. The serial administration and/or delivery of the indirect and direct immune modulating compositions can support or provide a synergistic and dynamic balance of the body's immune system. When the indirect and direct immune modulating compositions are administered or delivered (e.g., sequentially) separated by a predetermined time period or interval (e.g., a predetermined number of hours), the variation, regulation, alternation or fluctuation of immune cells supported, associated with, or caused by the compositions is expected to lead to or provide an enhanced health benefit, more particularly, an improved immune system function, which can support cancer cell reduction. Compositions in accordance with embodiments of the disclosure, whether in the form of a single time-release compound/delivery vehicle or separate formulations/separate delivery vehicles, are administered or delivered to or intended for absorption by the system of the organism (e.g., person or animal) at separate or distinct times or time intervals. This allows for separate or segregated absorption of the compositions.

The indirect and direct immune modulating compositions can be in various forms. Representative aspects of immune modulating compositions in accordance with embodiments of the present disclosure are described in detail hereafter.

### Representative Aspects of a Direct Immune Modulating Composition

The direct immune modulating composition can be in essentially any form, for example, a beverage; one or more capsules, tablets, or pills; a powder; a food composition; an injection (parenteral); and/or a dietary supplement, and the like. The direct immune modulating composition can be administered or introduced into a living organism at a first time or during a first time period.

In embodiments, the direct immune modulating composition supports, facilitates, or provides immune modulation, immune balancing, cancer cell reduction, and/or tumor cell reduction within the body of a living organism. The direct immune modulating compositions can include or incorporate one or more components (e.g., natural extracts corresponding to phytochemical compositions and/or nutraceutical compositions). More particularly, the direct immune modulating composition can include at least an extract from mangosteen and an extract from Gotu Kola to support, facilitate, or provide an immune modulation, immune balancing, cancer cell reduction, and/or tumor reduction within the living organism's body. Extracts of mangosteen include, but are not limited to, for example, xanthones such as α-mangostin, 8-desoxygartanin, gartanin, garcinone E, β-mangostin, 3-mangostin, γ-mangostin and 9-hydroxycalabaxanthone, and the like and combinations of these. Extracts of Gotu Kola include, but are not limited to, for example, oligoglycosides, centellasaponins, leutine, beta-carotene, triterpenoids, and the like, and combinations of these.

In some embodiments, the direct immune modulating compositions can include additional substances. For example, the direct immune modulating composition can include one or more of an extract from soybean (also known as Soya bean or Glycine Max), an extract from Sesamum indicum L. (hereinafter referred to as “sesame”), and an extract from Psidium Guajava L. (hereinafter referred to as “guava”) to further support, promote, or provide a therapeutic effect. Chemicals, for example additives, stabilizers, emulsifiers, and preservatives, or other natural compounds (e.g., phytochemicals) can be optionally added to the composition as required. Extracts of soybean can include, but are not limited to for example, isoflavones, lignans, glycine and the like, and combinations of these. Extracts of sesame can include, but are not limited to for example, vitamin E, magnesium, copper, calcium, iron, zinc, vitamin B6, sesamol, sesamin, and the like and combinations of these. Extracts of guava can include, but are not limited to for example, tannins, β-sitosterol, maslinic acid, essential oils, triterpenoids, flavonoids, alkaloids, saponins, cardenolides, and the like, and combinations of these.

Embodiments of the present disclosure include a direct immune modulating composition that includes specific concentrations or quantities (e.g., predetermined percentages by mass or weight of the compositions) of an extract from at least some of mangosteen, Gotu Kola, soybean, sesame, and/or guava, typically including at least an extract of mangosteen and an extracdt of Gotu Kola. In some embodiments, these extracts can therapeutically support, influence, or affect immune modulation, immune balancing, cancer cell reduction, and/or tumor cell reduction within the body of a living organism. In embodiments, the direct immune modulating composition can result in improved or enhanced immune system functions such as, for example, fighting off pathogens or foreign matter in the body and decreasing allergic and/or autoimmune reactions. The concentration or quantity (e.g., a predetermined percentage by weight of the composition) of the extract from mangosteen, Gotu Kola, soybean, sesame, and/or guava can vary depending upon embodiment details.

In embodiments, the direct immune modulating composition includes between approximately 1% and 99% (for example, approximately 5%-95%, or approximately 10%-90%) by mass, weight, or volume of a combination of at least two of the extract from mangosteen, the extract from Gotu Kola, the extract from sesame, the extract from soybean, and the extract from guava.

In embodiments, the direct immune modulating composition can include approximately 1%-100% (e.g., approximately 5%-95%, or approximately 10%-90%, or approximately 20%-80%, or approximately 25%-75%, or approximately 35%-65%, or approximately 50%) by mass, weight, or volume of a combination of the extract from mangosteen and the extract from Gotu Kola. Any corresponding portion of the composition relative to 100% (e.g., the total composition amount, mass, weight, or volume) can include one or more of extract from sesame, extract from soybean, extract from guava, and another substance (e.g., another phytochemical, or a solid or liquid carrier material or filler).

More particularly, in embodiments, the direct immune modulating composition can have a mass or volume defined as a 100% reference mass or volume that includes: a) approximately 10%-90% by mass or volume of a primary substance including extract from mangosteen and extract from Gotu Kola; and, b) a secondary or expected adjunctive substance in a percentage defined by the 100% reference mass or volume minus the percentage mass or volume of the primary substance, where the secondary substance includes extract from sesame, extract from soybean, extract from guava, and/or another extract or substance. In a representative example, approximately 40% of the composition can be made up of the primary substance, and approximately 60% of the composition can be made up of the secondary substance. In another representative example, approximately 50% of the composition can be formed from the primary substance, and approximately 50% of the composition can be formed from the secondary substance.

The direct immune modulating composition can support, facilitate, lead to, or provide the direct regulation or modulation of an immune system by way of inducing a change in the quantity of white blood cells, proteins and signaling molecules secreted by white blood cells within biological tissue (e.g., the body of the living organism). More specifically, a direct immune modulating composition according to an embodiment of the present disclosure can support, lead to, or provide the alteration, change, increase, or decrease in the number of white blood cells, such as, for example, phagocytes, Natural Killer cells (NK cells), cytotoxic T-cell, B cell, plasma cells, Tₕ cells, Tₕ-1 cells, Tₕ-2 cells, Tₕ-17 cells, and T_{reg} cells. Additionally, the direct immune modulating composition according to the present disclosure can support, facilitate, lead to, or provide the alteration, change, increase, or decrease in the amount or quantity of signaling molecules (cytokines) secreted by white blood cells including but not limited to, interferon (IFN-γ), tumor necrosis factor (TFN-α), and interleukin (IL) 2, 4, 5, 9, 10, 13, 17, and 22. In embodiments, the effect(s) associated with the direct immune modulating composition on the immune system can be called a “direct effect” as it raises particular cytokine levels.

In embodiments, the direct immune modulating composition can support, facilitate, lead to, or provide an increased expression of Tₕ-1 as determined by expression cytokines of TNF-α, INF-γ, and IL-2. In embodiments, the direct immune modulating composition can support, facilitate, lead to, or provide an increased, changed, or altered expression of TNF-α as compared to the level in a living organism taking a different non-pharmaceutical supplement by about 25% to about 40%. In embodiments, the direct immune modulating composition can increase expression of TNF-α by about 34.9%. The direct immune modulating composition can change, alter, or increase INF-γ expression as compared to a living organism taking a different non-pharmaceutical supplement by about 80% to about 90%. In embodiments, INF-γ expression can be increased by about 86%.

In some embodiments, the direct immune modulating composition can support, facilitate, lead to, or provide an increase, change, or alteration of expression of Tₕ-2 as determined by expression of cytokines IL-113, IL-6, IL-13, and IL-5. In embodiments, the direct immune modulating composition can increase expression of IL-6 by 45% to about 55% as compared to a living organism taking a different non-pharmaceutical composition or supplement. In some embodiments, the direct immune modulating composition can increase IL-6 expression by about 51%.

In some embodiments, the direct immune modulating composition can support, facilitate, lead to, or provide an increase, change, or alteration of expression of Tₕ-17 as determined by cytokine IL-17 levels. In embodiments, the direct immune modulating composition can support, facilitate, lead to, or provide an increase, change, or alteration of expression of IL-17 levels by about 370% to about 380% as compared to a living organism taking a different non-pharmaceutical supplement. In embodiments, the direct immune modulating composition can increase expression of IL-17 by about 376%.

In some embodiments, the direct immune modulating composition can support, facilitate, lead to, or provide an increase, change, or alteration of expression of T_{reg} as determined by cytokines IL-9 and IL-10 levels. In embodiments, the direct immune modulating composition can increase IL-9 levels by about 105% to about 115% as compared to a living organism taking a different non-pharmaceutical supplement. In embodiments, the direct immune modulating composition can increase expression of IL-9 by about 110%.

A daily dose useful to support, facilitate, lead to, or provide an increase, change, or alteration of expression and/or direct regulation or modulation of an immune system by way of inducing a change in the quantity of white blood cells, proteins and signaling molecules secreted by white blood cells within biological tissue (e.g., of an average size child or adult human being, or a reduced mass child or adult human being suffering from a health condition or immune dysfunction, although other living organisms can be treated) of the direct immune modulating composition in accordance with the present disclosure can provide at least approximately 10 μg of an extract of mangosteen and at least approximately 10 μg of an extract of Gotu Kola. In some embodiments, a direct immune modulating composition can include about 10 mg to about 250 mg extract of mangosteen per dosage unit (capsule, pill, syrup, etc.). In some embodiments, a daily dose of a composition formulated in accordance with the present disclosure provides at least approximately 0.1 mg to about 1 mg of each of an extract of mangosteen and an extract of Gotu Kola; or at least approximately 5 mg to about 1000 mg (e.g., about 25 to about 750 mg, or about 50 to about 500 mg, or about 100 mg to about 300 mg) of a combination of an extract of mangosteen and an extract of Gotu Kola. Any daily dose can be divided into sub-doses, for administration in accordance with the method herein disclosed, based upon time of day, meal consumption, and/or health condition. For example, a daily dose can be divided and administered in a form of capsules by consuming, for example, 4 capsules each containing ¼ of the total dosage, the capsules including an extract of mangosteen and Gotu Kola, daily. It will however be understood by a person of ordinary skill in the art that alternative dosages (e.g., 3, 5, 6, or 9 capsules per day) can be suggested or used.

### Representative Aspects of an Indirect Immune Modulating Composition

The indirect immune modulating composition can be in essentially any form, for example, a beverage; one or more capsules, tablets, or pills; a powder; a food composition; an injection (parenteral); and/or a dietary supplement, and the like. The indirect immune modulating composition can be administered or introduced into a living organism at a first time or during a first time period (e.g. an indirect composition administration time or time period). Administration of the indirect immune modulating composition can be at a separate time or time period from that of the direct immune modulating composition.

In embodiments, the indirect immune modulating composition supports, facilitates, or provides immune modulation, immune balancing, cancer cell reduction, and/or tumor cell reduction within the body of a living organism. In embodiments, the effect(s) associated with the indirect immune modulating composition on the immune system can be called an “indirect effect” as it does not raise cytokine levels in the same fashion as a direct immune modulating composition.

The indirect immune modulating compositions can include or incorporate one or more components (e.g., phytochemical compositions and nutraceutical compositions). More particularly, the indirect immune modulating composition can contain an extract of mangosteen, alone or in combination with other particular active ingredients or compounds that can support or facilitate tumor or cancer cell reduction, such as: alpha hydroxyacids which bind to free ferrous ions that may otherwise be used by cancer cells to accelerate multiplication process; hydroxy citric acid (HCA), which is believed to bind to an enzyme that cancer cells require for lipid synthesis or production as a component of cancer cell membrane; GM-1, an active xanthone that is believed to directly kill cancer cells when contacted therewith; and/or polysaccharides, believed to be potent immunomodulators some of which have been shown to have direct cytotoxic effects on tumor cells. In various embodiments, indirect immune modulating compositions exclude or essentially exclude an extract of Gotu Kola corresponding to the direct immune modulating compositions.

In some embodiments, the indirect immune modulating composition can function without elevating levels of immune cells. In embodiments, the indirect immune modulating composition supports, facilitates, or provides modulation to immune cells. In some embodiments, the indirect immune modulating composition can decrease levels of certain cytokines, such as, for example, TNF-α, INF-γ, IL-1β, IL-6, and/or IL-17. In some embodiments, the indirect immune modulating composition can increase levels of certain cytokines, such as, for example IL-9 and/or IL-10.

In embodiments, the indirect immune modulating composition can include mangosteen. In embodiments, the indirect immune modulating composition can include xanthones from mangosteen standardized to from about 10 mg to about 250 mg. In embodiments, the indirect immune modulating composition can include xanthones from mangosteen standardized to about 100 milligram of xanthones per dosage. It will however be understood by a person of ordinary skill in the art that alternative dosages can be suggested or used.

In embodiments, the indirect immune modulating composition can be in the form of a beverage that includes mangosteen extract according to the present disclosure. The mangosteen extract of the indirect immune modulating composition can include, for example, α-hydroxyacids, hydroxy citric acid (HCA), polysaccharides, GM-1, other xanthones, and combinations of these.

Therefore, in embodiments, the indirect immune modulating composition, according to the present disclosure, can support or facilitate cancer cell reduction and aid the immune system without involving direct immune modulation. In embodiments, the indirect immune modulating composition can support or facilitate reduction of inflammatory symptoms caused by elevated levels of certain immune cytokines in, for example, autoimmune disease or allergic reactions. In some embodiments, the activity of the indirect immune modulating composition can be “indirect,” as the indirect immune modulating composition can support or assist the immune system without elevating levels of some cytokines, such as, for example IL-17, IL-13, IL-6, IL-1β, IFN-γ, TNF-α, and IL-2.

In embodiments, the indirect immune modulating composition can decrease levels of Tₕ-1, Tₕ-2, and Tₕ-17, while increasing levels of T_{reg} cells. In embodiments, the indirect immune modulating composition can decrease Tₕ-1 cells as determined by levels of cytokines TNF-α, INF-γ, and IL-2 by from about 5% to about 20%. In embodiments, the indirect immune modulating composition can decrease levels of Tₕ-2, as determined by the levels of cytokines IL-113, IL-6, and IL-13 by from about 20% to about 45%. In embodiments, the indirect immune modulating composition can increase levels of T_{reg} cells as determined by levels of cytokines IL-9 and IL-10 by from about 5% to about 55%.

### Administration

When a set or series of compositions including a set of direct immune modulating compositions and a set of indirect immune modulating compositions are administered, the antipode or different mode of action of each of the compositions can support or provide an enhanced or synergistic health benefit relating to an immune modulation and support or facilitate cancer cell reduction. The direct immune modulating composition(s) and indirect immune modulating composition(s) can be administered so as to increase or support the increase of one or more cytokine levels, or decrease or support the decrease of one or more cytokine levels, or provide components that support, lead to, induce or facilitate tumor or cancer cell reduction by way of supporting or aiding the immune system.

Fluctuating modulation of the immune system can be provided by the segregated administration, delivery, or absorption of the direct and indirect immune modulating compositions, for instance by way of administering or delivering the direct and indirect compositions in accordance with a regimen or protocol associated with or defining a set of direct composition administration times (e.g., one or more times per day) and a set of indirect composition administration times (e.g., once per day). The direct and indirect compositions support, affect, or act on the immune system in different ways. In embodiments, the direct immune modulating composition can result in elevation of the level of Tₕ-17 cells, to support or facilitate cancer cell reduction. While in embodiments, the indirect immune modulating composition can support or facilitate cancer cell reduction without associating with a regulation of a number, quantity, or amount of immune cells. In embodiments, the indirect immune modulating composition can support, lead to, or induce, facilitate, or modulate aspects of immune system function by inducing a decrease in the number of certain white blood cells so as to allow other components of the indirect immune modulating composition to affect or act on cells within the body without initiating an elevated immune response.

In accordance with embodiments of the present disclosure, administration of direct and indirect compositions in a segregated manner or in series is associated with the direct immune modulating composition supporting, leading to, inducing, facilitating, or altering immune cell quantity, and the indirect immune modulating composition providing a different mode of action than the direct immune modulating composition to support, lead to, induce, facilitate or provide enhanced, synergistic, health benefits. In several embodiments, the direct immune modulating composition can be administered at, within, or across a first or direct administration time or time interval, and the indirect immune modulating composition can be administered at a second or indirect administration time which is separate from the first or direct administration time or time interval.

In some embodiments, the administration of the direct immune modulating composition and the indirect immune modulating composition can be separated by, for example, about 2 hours to about 6 hours. In embodiments, administration of the direct immune modulating composition and the indirect immune modulating composition is separated by about 3 hours. The compositions in accordance with embodiments of the present disclosure, whether in the form of a single time-release compound or separate formulations, are administered (or delivered/released) to the system of the organism (person/animal) at separate times to facilitate separate or independent absorption of the compositions. Thus, if the direct immune modulating composition is administered first, the composition can elevate certain cytokine levels, and then the indirect immune modulating composition can be administered at a later time to decrease certain cytokine levels to prevent overexposure or support or facilitate tumor or cancer cell reduction by aiding the immune system without involving direct immune modulation.

In some embodiments, a single time-release compound having both an immediate release portion and one or more delayed release portions can be administered having immune modulating activity, wherein the immediate release portion of the composition and the delayed release portion of the composition induce antipode reactions in the immune system. In embodiments, a single time-release compound can include an immediate release portion containing an indirect immune modulating composition, and multiple (e.g., about 2 to about 4) delayed-release portions containing at least a direct immune modulating composition, where the delayed-release portions each release at about 2 to about 6 hour intervals from each other. In embodiments including more than one delayed release portion, the delayed release portions can further include alternating serial releases of an indirect immune modulating composition and at least one direct immune modulating composition.

As stated above, embodiments of the present disclosure can include compositions that affect or support processes that affect white blood cell quantity. For instance, the compositions can facilitate or effectuate a change in quantity of white blood cells, proteins and signaling molecules secreted by white blood cells within biological tissue (e.g., the body of the living organism when consumed thereby). Compositions according to the present disclosure can support or facilitate an alteration, change, increase, or decrease in the number or conditions of white blood cells, including but not limited to phagocytes, Natural Killer cells (NK cells), cytoxic T-cell, B cell, plasma cells, Tₕ cells, Tₕ-1 cells, Tₕ-2 cells, Tₕ-17 cells, and T_{reg} cells. Compositions according to the present disclosure can support or facilitate the alteration, change, increase, or decrease in the amount or quantity of signaling molecules (cytokines) secreted by white blood cells including but not limited to IFN-γ, TFN-α, IL-2, IL-4, IL-5, IL-9, IL-10, IL-13, IL-17, and IL-22.

Additionally, administration of these compositions in a serial manner can improve the self-reported quality of life of those suffering from cancer or other forms of tumorgenesis. Quality of life can be measured or recorded, for example, by an individual experiencing an improvement or physiological improvement such as going from being bedridden to capable of sitting, to capable of standing, to capable of walking, and the like. In embodiments, use of the compositions in accordance with embodiments of the present disclosure can provide prolongation of quality of life for those suffering from late-stage or end-stage cancer. In embodiments, administration of the compositions of the present disclosure can support or facilitate a reduction in biomarker levels used to detect cancer activity. Biomarkers include, for example, alpha-fetoprotein (AFP), prostate specific antigen (PSA), carcinoembryogenic antigen (CEA), lactic acid dehydrogenase (LDH), and the like and combinations of these. Biomarker levels can be reduced, for example, from levels elevated by cancer toward or to normal (healthy adult) range for the biomarker.

Representative examples of compositions provided by the present disclosure are described in the examples below. It will be understood by a person of ordinary skill in the art that the scope of the present disclosure is not limited to the following examples.

### EXAMPLES

### Example 1

A direct immune modulating composition in the form of a capsule was provided in accordance with the present disclosure. The capsule included 25% by weight mangosteen extract, 25% by weight of Gotu Kola extract, 16.67% of sesame extract, 16.67% of soybean extract, and 16.67% of guava extract. The capsules were formulated or manufactured by standard methods, processes, and/or techniques known to a person of ordinary skill in the art.

The capsules were administered to human volunteers in a dosage of 4 capsules per day for 15 days. Six other human volunteers were provided a dosage of 4 placebo capsules per day for 15 days.

On day 15, blood cytokine levels were tested using flow cytometry (FlowCytomix™) Levels of IL-2, TNF-α, and IFN-γ indicative of the amount of Tₕ-1 cells; IL-113, IL-6, IL-13 and IL-5 indicative of the level of Tₕ-2 cells and IL-17/IL-22 and IL-9/IL-10 indicative of the amount of Tₕ-17 and T_{reg} cells, respectively were tested.

### Results

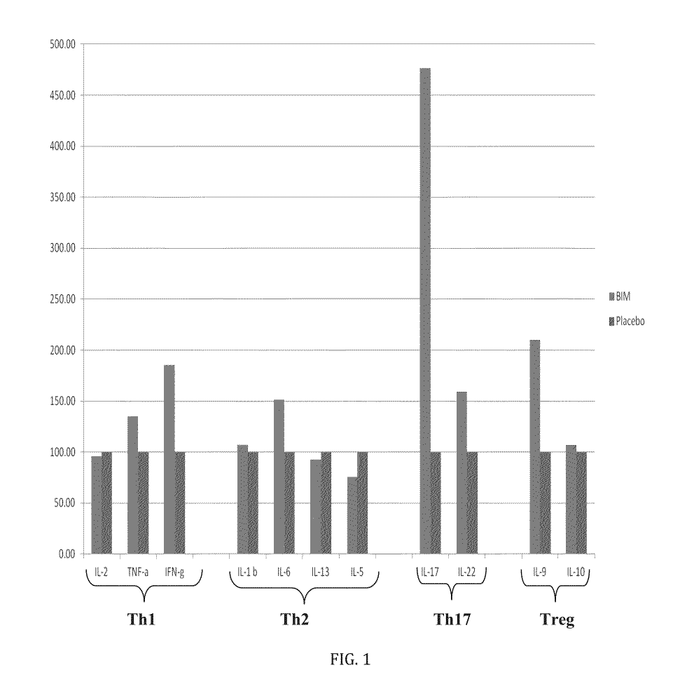

FIG. 1 shows an effect of a composition, identified as BIM (“Balanced Immune Modulating” composition) in FIG. 1, according to present disclosure comprising an extract from mangosteen, Gotu Kola, sesame, soybean, and guava on the level of Tₕ-1, Tₕ-2, Tₕ-17, and T_{reg} cells, indicative of immune modulation. Placebo or reference composition results were normalized to 100. Upon administration of the direct immune modulating composition, the amount of Tₕ-1 cells increased, as compared to a placebo or reference group. The amount of Tₕ-2 cells in the volunteer group remained unchanged as compared to the placebo group. Elevation of Tₕ-1 cells in conjunction with unchanged Tₕ-2 cell number indicates that the direct immune modulating composition can support, facilitate, or provide immune modulation, or immune balancing.

Furthermore, as illustrated in FIG. 1, the quantity of Tₕ-17 in volunteers taking the direct immune modulating composition according to the present disclosure increased by five fold, as compared to a placebo group. This result suggests that the direct immune modulating composition can facilitate or effectuate immune modulation by inducing a five-fold increase of Tₕ-17 cells, which are capable of fighting pathogens or foreign matter beyond Tₕ-1 and Tₕ-2 cell capability. In other words, the direct immune modulating composition provided by the present disclosure can support, improve, or enhance immune system function to fight excess pathogens or foreign matters that escape from Tₕ-1 and Tₕ-2.

The direct immune modulating composition provided by the present disclosure surprisingly appears to increase Tₕ-17 cells approximately five fold. Thus, the results obtained in Example 1 suggest that the direct immune modulating composition according to the present disclosure can support or facilitate cancer cell reduction. The level of T_{reg} cells in volunteers taking the direct immune modulating composition provided by the present disclosure is higher than a placebo group.

### Conclusion

Experiments in Example 1 indicate that the direct immune modulating composition provided by the present disclosure can support, facilitate, or provide immune modulation or immune balancing specifically of Tₕ-1, Tₕ-2, Tₕ-17, and T_{reg} cells. In addition, the immune modulating composition according to the present disclosure can support or facilitate cancer cell reduction through a regulation or modulation of Tₕ-17 cells.

### Example 2

An indirect immune modulating composition in the form of a beverage, a liquid admixture, or a drink was provided in accordance with the present disclosure. The beverage included mangosteen extract. The mangosteen extract beverage was formulated or manufactured by standard methods, processes, and/or techniques known to a person of ordinary skill in the art.

A dosage of one bottle of mangosteen beverage per day containing at least 100 milligram of xanthones was administered to volunteers. The beverage containing an extract from mangosteen was administered to six volunteers for the duration of 15 days while six volunteers received a placebo or reference beverage.

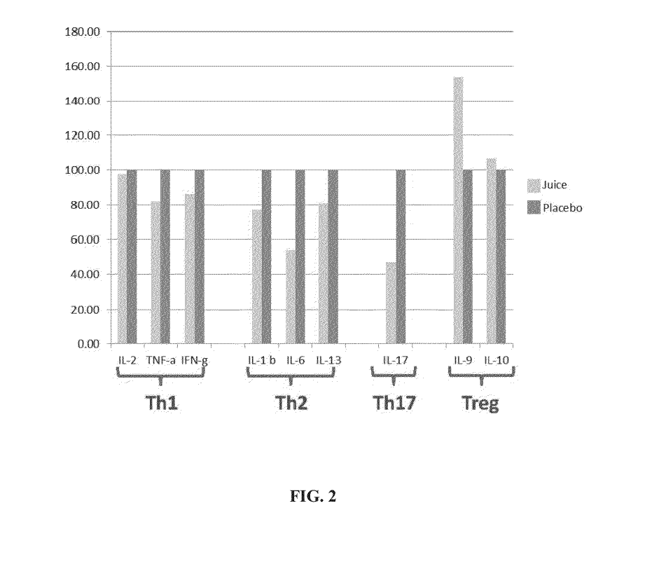

On day 15, blood cytokine levels were tested using flow cytometry (FlowCytomix™) Levels of IL-2, TNF-α, and IFN-γ indicative of the amount of Tₕ-1 cells; IL-1β, IL-6, IL-13 and IL-5 indicative of the amount of Tₕ-2 cells; and, IL-17 and IL-9/10 indicative of the amount of Tₕ-17 and T_{reg} cells, respectively were tested. The results are depicted in FIG. 2, where the placebo or reference beverage results have been normalized to 100.

### Results

FIG. 2 shows an effect of the indirect immune modulating composition according to present disclosure on the level of Tₕ-1, Tₕ-2, Tₕ-17, and T_{reg} cells. Upon administration of the indirect immune modulating composition the level of Tₕ-1 cells remained relatively unchanged or insignificantly decreased. The level of Tₕ-2 cells in the volunteer group decreased as compared to a placebo or reference group.

As further illustrated by FIG. 2, the level of Tₕ-17 decreased in volunteers taking the indirect immune modulating composition. The level of T_{reg} cells in volunteers taking the second immune modulating composition provided was higher than the placebo or reference group. Comparatively, an increase of T_{reg} cells upon a consumption of the composition provided by the present disclosure causes a reduction of Tₕ-1 cells, a more noticeable reduction of Tₕ-2 cells, and even more significant reduction of Tₕ-17 cells.

### Conclusion

Experiments in example two indicated that the indirect immune modulating composition provided by the present disclosure, in the form of liquid containing an extract from mangosteen, can support or facilitate the modulation of immune cell levels, specifically the levels of Tₕ-1, Tₕ-2, Tₕ-17, and T_{reg} cells.

### Example 3

The direct and indirect immune modulating compositions provided by the present disclosure were administered serially at a predetermined dosage and at predetermined intervals. The indirect immune modulating composition in the form of a beverage comprising an extract from mangosteen was initially administered or introduced into a person in the morning. A direct immune modulating composition in the form of a capsule including 25% by weight of an extract from mangosteen, 25% by weight of an extract from Gotu Kola, 16.67% of an extract from sesame, 16.67% of an extract from soybean, and 16.67% of an extract from guava was subsequently administered in the afternoon, evening, and before bedtime.

### Example 4

A trial of 17 lung cancer patients, each having a life expectancy of 2-3 months, was undertaken. None of the patients had responded to rounds of chemotherapy.

The trial length has varied based on time of entry into the study and continues at the time of filing. Patients have been taking the compounds for as long as 20 months and as few as 6 months.

Each of the human subjects was administered a series of substances in the manner described in Example 3 above (i.e. juice in the morning and 3 capsules at each of the afternoon, evening, and before bedtime).

All 17 human lung cancer patients are still living and can now walk as compared to before joining the program, and some have shown white blood cell increases, such as Th-1 increase. Additionally, all of the cancer patients report enhanced quality of life as compared to before treatment.

### Example 5

Four human patients with cancer were administered a series of substances in the manner described in Example 3 above (i.e. juice in the morning and 3 capsules at each of the afternoon, evening, and before bedtime). Conditions and responses to therapy are detailed below for each patient and outlined in Table 1 below.

### Patient 1

Male, age 65

Condition: Sinus cancer metastasis to lung, lymph nodes and bone.

Onset: 1994

Previous Treatments: traditional and laser surgeries, post-operative follow-up.

Symptoms: chronic cold symptoms

Began Treatment: 2009

Treatment: daily regime per Example 3 (ongoing at the time of filing the application) with two bottles indirect immune modulating composition in the morning followed by two capsules three times per day of direct immune modulating composition at 2 to 6 hour intervals following the indirect immune modulating composition.

Markers: Carcinoembryogenic antigen (CEA) [Normal for adult non-smoker is less than about 2.5 ng/ml; adult smoker less than about 5.0 ng/ml]
Pre-treatment with method of disclosure CEA level range=18 to 50 ng/ml
Two months post treatment: 3.8 ng/ml
Four months post treatment: 1.9 ng/ml
Six months post treatment: 1.2 ng/ml

PET Scan Pre treatment: showed cancer in sinuses, lungs, bone and lymph nodes.

PET Scan Four months Post treatment: normal.

PET Scan at time of application filing: normal.

### Patient 2

Male

Condition: Liver cancer metastasis to lung, lymph nodes, and bone

Previous treatment: Surgery to remove 2.4 cm×1.8 cm×3 cm liver tumor Treatment: One bottle indirect immune modulating composition followed by 3 capsules direct immune modulating composition three times per day at intervals of between about 2 and 6 hours.

Marker: alpha-fetoprotein (AFP) [Healthy adults <10 ng/ml, elevated with pregnancy, hepatitis, cirrhosis, cancer]
Pre-treatment: 129 ng/ml
24 months treatment: less than 1 ng/ml
CT Scan at 28 months treatment: no black spots on liver, clean scan

### Patent 3

Male, age 63

Condition: late stage prostate cancer

Previous Treatment: surgery followed by ten days radiation

Treatment: 1 bottle indirect immune modulating composition followed by three capsules direct immune modulating composition three times per day at intervals of between about 2 and 6 hours.

Marker: Prostate specific antigen (PSA) [healthy adult less than 4.0 ng/ml although cancer has been found at lower levels]
Pre-treatment: 100 ng/ml
32 days treatment: 31.88 ng/ml
95 days treatment: 1.8 ng/ml
200 days treatment: 0.82 ng/ml
284 days treatment: 0.87 ng/ml

### Patient 4

Female, age 48

Condition: Stage 4a lymph node cancer metastasis to bone causing femur fracture, weight loss, pain

Previous Treatments: 8 rounds of chemotherapy, bone graft surgery

Treatment: 1 bottle indirect immune modulating composition followed by 2 capsules direct immune modulating composition three times per day at about 2 to about 6 hour intervals.

Marker: Lactic acid dehydrogenase (LDH) [Healthy adult range from 140 units (U)/L to about 330 U/L, although the range can vary depending on the laboratory testing the sample]
1 year treatment: 300-320 U/L
Bone regeneration at femur fracture
Weight gain of 3 kg

TABLE 1

| | Patient No. | | | |
|---|---|---|---|---|
| DATA | NO. 1 | NO. 2 | NO. 3 | NO. 4 |
| Sex | M | M | M | F |
| Age | 65 | 67 | 63 | 48 |
| Cancer | Nasal | Liver | Prostate | Lymph Node |
| Metastasis | Lung, lymph | Liver | Body, Bone, Spinal | Bone |
| | nodes, bone | | Court | |
| Prior | Traditional | Surgery | Surgery/radiation | Eight rounds |
| Treatment | surgery/laser | | | chemotherapy/ |
| | surgery | | | surgery |
| Symptoms | Chronic cold | weakness | Weakness, weight loss, | Weight loss, pain |
| | | | pain, paralysis in the | |
| | | | lower part of body (from | |
| | | | hip down) | |
| Marker | CEA level: 18- | AFP level: | PSA level: 100 ng/ml | LDH |
| Pre | 50 ng/ml | 129 ng/ml | | |
| Marker | 3.8/1.9/1.2 | <1 ng/ml | 31.88/1.86/0.82/0.87 | 300-320 μ/l |
| Post | ng/ml | | | |

The administration regime as described in Example 3 supports or provides a dynamic balance of the immune system by way of fluctuating modulation. When the direct and indirect immune modulating compositions are administered in series at predetermined intervals, the variation, regulation, alternation or fluctuation of immune cells and immune function supported by, associated with, or provided by the administered immune modulating compositions can synergistically provide surprisingly enhanced health benefit or improved immune system, and can support or facilitate significant or dramatic cancer cell reduction.

Particular embodiments of the disclosure are described above for providing novel, inventive, advantageous, synergistic, and/or enhanced methods of administration and compositions for improving, enhancing, or facilitating immune system activity as well as supporting or facilitating cancer cell reduction. While methods, compositions, applications, techniques, advantages, features and alternatives associated with certain embodiments have been described within the context of those embodiments, other embodiments can also exhibit such advantages, and not all embodiments need necessarily exhibit such advantages to fall within the scope of the disclosure. It will be appreciated that several of the above-disclosed methods, features, functions, compositions, applications, techniques, advantages, and alternatives thereof, can be desirably combined into other methods, compositions or applications. The above-disclosed methods, functions, compositions, applications, techniques, advantages, and alternatives thereof, as well as various presently unforeseen or unanticipated alternatives, modifications, variations or improvements thereto that can be subsequently made by one of ordinary skill in the art, are encompassed by the following claims.

1. A method of supporting or providing fluctuating modulation to immune function comprising:
administering a set of direct immune modulating compositions; and
administering an indirect immune modulating composition,
wherein the administration of the set of direct immune modulating compositions and the administration of the indirect immune modulating composition is separated by about 2 to about 6 hours.

2. The method according to claim 1, wherein said indirect immune modulating composition results in antipode effects to said set of direct immune modulating compositions with respect to at least one of TNF-α, INF-γ, IL-6, and IL-17.

3. The method according to claim 1, wherein the set of direct immune modulating compositions comprises an extract of Gotu Kola and at least one extract of substances selected from the group consisting of sesame, soybean, guava and mangosteen.

4. The method according to claim 1, wherein the indirect immune modulating composition comprises extract of Garcina mangostana.

5. The method according to claim 4, wherein the extract of Garcinia mangostana is selected from the group consisting of α-hydroxyacids, hydroxy citric acid, GM-1, xanthones, and polysaccharides.

6. The method according to claim 4, wherein the indirect immune modulating composition excludes extract of Gotu Kola.

7. The method according to claim 2, wherein the set of direct immune modulating compositions results in an increase of one or more cytokines selected from the group consisting of TNF-α, IFN-γ, IL-1β, IL-6, IL-17, IL-9, and IL-10.

8. The method according to claim 2, wherein the indirect immune modulating composition results in a decrease of one or more cytokines selected from the group consisting of TNF-α, IFN-γ, IL-1β, IL-6, IL-17 and IL-13.

9. The method of claim 1, wherein the set of direct immune modulating compositions and the indirect immune modulating composition are contained in a single time-release capsule.

10. The method of claim 1, wherein the set of direct immune modulating compositions result in elevated cytokine levels and the indirect immune modulating composition results in indirect modulation of the immune system.

11. An immune modulating time-release compound comprising:
an immediate release portion comprising an indirect immune modulating composition; and
at least one delayed release portion comprising a set of direct immune modulating compositions.

12. The immune modulating time-release compound of claim 11, wherein the indirect immune modulating composition comprises xanthones.

13. The immune modulating time-release compound of claim 12, wherein the amount of xanthones is from about 10 mg to about 250 mg.

14. The immune modulating time-release compound of claim 12, wherein the indirect immune modulating composition excludes Gotu Kola.

15. The immune modulating time-release compound of claim 11, wherein the set of direct immune modulating compositions comprises Gotu Kola extract.

16. The immune modulating time-release compound of claim 15, wherein the set of direct immune modulating compositions further comprises an extract of at least one of mangosteen, soybean, sesame, and guava.

17. The immune modulating time-release compound of claim 11, wherein the delayed release portion releases about 2 hours to about 6 hours after the immediate release portion.

18. The immune modulating time-release compound of claim 15, wherein said at least one delayed release portion includes 2 to 4 delayed release portions, said delayed release portions releasing at about 2 to about 6 hour intervals.

19. A kit comprising:
an indirect immune modulating composition in a first administration form; and
a direct immune modulating composition in a second administration form, wherein the indirect immune modulating composition and the direct immune composition result in antipode effects upon an immune system of an organism to which the indirect immune modulating composition and the direct immune modulating composition are administered.

20. The kit of claim 19, wherein the indirect immune modulating composition comprises an extract of mangosteen and excludes an extract of Gotu Kola, and wherein the direct immune modulating composition includes a combination of an extract of mangosteen and an extract of Gotu Kola.

21. The kit of claim 19, wherein the first administration form comprises a liquid, and the second administration form comprises a set of capsules.

22. The kit of claim 19, further comprising dosage and administration instructions defining an indirect immune composition administration time relative to at least one direct immune modulating composition administration time that is separate from the indirect immune composition administration time.

23. The kit of claim 22, wherein the indirect immune modulating composition administration time and the direct immune modulating composition time are separated by at least approximately 2 hours.

24. A method for reducing biomarker levels associated with cancer comprising:
administering an indirect immune modulating composition in the form of a liquid comprising xanthones at a first time point; and,
administering a direct immune modulating composition in the form of capsules comprising Gotu Kola, and at least one of sesame, soybean, guava and mangosteen at a second time point;
wherein the first time point and the second time point are separated by about 2 to about 6 hours.
